(19) United States
(12) Patent Application Publication
(10) Pub. No.: US 2023/0127784 A1
(43) Pub. Date: Apr. 27, 2023

(54) DEHYDROGENATION CATALYST FOR PRODUCTION OF OLEFINS FROM ALKANE GASES AND PREPARATION METHOD THEREOF

(71) Applicants: SK GAS CO., LTD., Seongnam-si, Gyeonggi-do (KR); KOREA RESEARCH INSTITUTE OF CHEMICAL TECHNOLOGY, Daejeon (KR)

(72) Inventors: Daesung PARK, Daejeon (KR); Hawon PARK, Daejeon (KR); Changyeol SONG, Daejeon (KR); Yong Ki PARK, Seoul (KR); Won Choon CHOI, Daejeon (KR); Ung Gi HONG, Seongnam-si, Gyeonggi-do (KR); Hae Bin SHIN, Seongnam-si, Gyeonggi-do (KR); Miyoung LEE, Seongnam-si, Gyeonggi-do (KR); Deuk Soo PARK, Seongnam-si, Gyeonggi-do (KR)

(73) Assignees: SK GAS CO., LTD., Seongnam-si, Gyeonggi-do (KR); KOREA RESEARCH INSTITUTE OF CHEMICAL TECHNOLOGY, Daejeon (KR)

(21) Appl. No.: 17/909,241

(22) PCT Filed: Dec. 24, 2020
(86) PCT No.: PCT/KR2020/019141

§ 371 Date: Sep. 2, 2022

(30) Foreign Application Priority Data
Mar. 10, 2020 (KR) 10-2020-0029648

(51) Int. Cl. B01J 21/04; B01J 23/06; B01J 23/75; B01J 37/02; C07C 5/333

(52) U.S. Cl. CPC B01J 21/04; B01J 23/06; B01J 23/75; B01J 37/0201; C07C 5/333

(57) ABSTRACT

The present disclosure is to provide a catalyst for olefin production which is eco-friendly and has excellent conversion rates and selectivity and a preparation method thereof, and the catalyst for olefin production according to the present disclosure is one in which cobalt and zinc are supported with alumina. Particularly, the catalyst according to the present disclosure uses an amount of platinum that is about 400 times smaller than that of the conventional catalysts, and has high conversion rates and selectivity under conditions in which continuous reaction-regeneration process is possible without an additional hydrogen reduction process.

### CROSS-REFERENCE TO PRIOR APPLICATIONS

This application is a National Stage Patent Application of PCT International Patent Application No. PCT/KR2020/019141 (filed on Dec. 24, 2020) under 35 U.S.C. § 371, which claims priority to Korean Patent Application No. 10-2020-0029648 (filed on Mar. 10, 2020), which are all hereby incorporated by reference in their entirety.

### BACKGROUND

The present disclosure relates to a catalyst for olefin production with improved selectivity and conversion rates compared to conventional technology in the production of olefins from alkane gases such as ethane, propane, butane, and the like, and a preparation method thereof.

Olefins such as ethylene and propylene are being widely used in the petrochemical industry. Typically, these olefins are obtained in the pyrolysis process of naphtha. However, since larger amounts of olefins are required in the petrochemical industry, olefins are also produced even through the dehydrogenation process of lower hydrocarbons using a catalyst.

A typical commercial process of the existing propane dehydrogenation (PDH) is a fixed bed reactor and a moving bed reactor.

On the other hand, there have been no commercialization cases to date in PDH technology (FPDH, Fast-fluidized propane dehydrogenation) using a fast-fluidized bed (hereinafter, fluidized bed) reactor.

The biggest difference between the fixed bed reactor and the fluidized bed reactor is a contact time of the catalyst and the reactant (propane). In other words, the fluidized bed reactor is a process in which propane is injected together with the catalyst into the fluidized bed reactor at a very high rate to react, and then the catalyst flows to the regeneration unit, and the product flows to the separation unit.

The goal of the FPDH process that has conventionally been developed is to have a residence time of the catalyst to 10 seconds or less. The shorter the residence time of the catalyst, the faster the injection speed of the propane supply amount. Since the catalyst is immediately regenerated and participates in the reaction again, production rate of propylene is significantly increased compared to the fixed bed process when it is developed as a commercial process.

However, since the contact time of the catalyst and propane is that short, the efficiency of the catalyst becomes very important. That is, it is important to maximize selectivity and conversion rate, which are two efficiency measures of the catalyst.

Furthermore, since the currently used propane dehydrogenation process technologies are composed based on noble metal catalysts or discontinuous processes, there are difficulties in propylene production operation, such as a reactor clogging phenomenon due to excessive activity of noble metal catalysts (coke generation) or fixed bed reactor valve sequence troubles.

Further, the propane dehydrogenation reaction has a limitation thermodynamically in the propane conversion rate due to the reversible reaction by hydrogen, and in order to overcome such a problem, hydrogen is converted into water using an external oxidant such as oxygen, halogens, sulfur compounds, carbon dioxide, water vapor, etc. in most processes.

Therefore, it is required to develop a new propane dehydrogenation process which solves the problem of the continuous process for effective mass production of propylene, and reduces production cost using a direct dehydrogenation catalyst without an oxidant.

Among the catalysts used for propane dehydrogenation, the reaction proceeds with a direct dehydrogenation mechanism in which hydrogen is adsorbed to active sites in the case of noble metal catalysts. However, the mechanism has not been clearly elucidated due to incompleteness of the active sites caused by electron mobility in the case of transition metal oxides.

Under these circumstances, most commonly used catalysts as PDH catalysts include Pt—Sn, VOₓ, and CrOₓ catalysts. Although the CrOₓ catalyst is very excellent in terms of propane conversion rate and selectivity, its use is limited due to problems such as environmental pollution and human hazards, and difficulties in controlling the oxidation reaction in the initial stage of the reaction. Platinum catalysts have excellent selectivity, but they are expensive and produce coke rapidly, so that fine control thereof is required. Further, the intrinsic activity of the catalyst varies depending on the combination of Sn, which is a co-catalyst component, and other metals, and due to the increase in the environmental hazard of Sn, the development of new multi-component catalysts for platinum catalysts is also continuously required.

Also, a Pt—Sn-based catalyst has been used in the conventional platinum-based dehydrogenation catalyst process, and it is known to contain approximately 0.4 wt. % (4,000 ppm) of platinum. FIG. 1 shows results of testing a Pt—Sn catalyst supported in a similar amount under the FPDH condition, which is a fluidized bed circulation process. Looking at the catalyst activity after performing regeneration with air, it can be seen that the initial conversion rate is 100%, but this is due to a side reaction that produces side products such as methane, carbon monoxide and ethane. When the hydrogen reduction pretreatment process was performed about 1 hour before the reaction, it showed a conversion rate of 51% and a propylene selectivity of 87% in about 5 seconds, which are levels to be applied to the FPDH process.

Meanwhile, in the case of Patent Documents 1 and 2, as techniques for a Zn—Pt-based catalyst, excessive platinum is used, and a reduction process is essentially used.

Accordingly, the present inventors have developed a catalyst for olefin production having both excellent catalyst conversion rates and selectivity compared to the conventional art by introducing a new catalyst containing a very small amount of platinum through continuous research, and a preparation method thereof.
(Patent Document 1) Japanese Patent No. 3908314
(Patent Document 2) Chinese Patent No. 105438568

### SUMMARY

An object of the present disclosure is to provide a catalyst for olefin production with excellent conversion rates and selectivity in the production of olefins from alkane gases such as ethane, propane, butane, and the like, and a preparation method thereof.

A catalyst for the production of olefins from alkane gases, according to the present disclosure, is one in which cobalt, zinc and platinum precursor solutions are co-impregnated and supported on alumina.

The catalyst calcination temperature is preferably 700° C. to 900° C.

It is preferable that cobalt is supported in an amount of 1 to 5% by weight based on the total catalyst weight.

It is preferable that zinc is supported in an amount of 2 to 10% by weight based on the total catalyst weight.

It is preferable that platinum is supported in an amount of 0.001 to 0.05% by weight based on the total catalyst weight.

It is preferable that a preparation method of a catalyst for the production of olefins from alkane gases, according to the present disclosure, comprises the steps of:

preparing a mixed solution by mixing cobalt, zinc and platinum precursors with water;

preparing a supported catalyst by impregnating the mixed solution on alumina;

drying the supported catalyst; and

calcinating the dried supported catalyst at 700° C. to 900° C.

It is preferable that another preparation method of a catalyst for the production of olefins from alkane gases, according to the present disclosure, comprises the steps of: preparing a mixed solution by mixing cobalt and zinc precursors with water;

preparing a supported catalyst A by impregnating the mixed solution on alumina;

preparing a platinum precursor solution;

preparing a supported catalyst B by impregnating the platinum precursor solution on supported catalyst A;

drying the supported catalyst B; and

calcinating the dried supported catalyst B at 700° C. to 900° C.

Another aspect of the present disclosure is to provide a method for producing continuous reaction-regenerated olefins containing a catalyst for the production of olefins from alkane gases produced according to the present disclosure.

In the method for producing continuous reaction-regenerated olefins, the reaction temperature is preferably 560 to 620° C.

In the method for producing continuous reaction-regenerated olefins, it is preferable that alkanes as a raw material have a flow rate (WHSV) of 4 to 16 h⁻¹.

The catalyst for the production of olefins from alkane gases such as ethane, propane, butane, and the like, according to the present disclosure, and the preparation method thereof have excellent conversion rates and selectivity, and thus are effective in both fixed-bed reactors and fluidized-bed reactors, but they particularly enable the realization of the FPDH process, which has not been previously commercially realized. Particularly, the catalyst according to the present disclosure uses platinum in an amount of about 400 times less than conventional catalysts, and has high conversion rates and selectivity under conditions in which continuous reaction-regeneration is possible without an additional hydrogen reduction process.

### BRIEF DESCRIPTION OF THE DRAWINGS

FIG. 1 schematically shows results of performing experiment of a Pt—Sn catalyst containing 0.42% by weight of platinum under FPDH conditions, a fluidized-bed circulation process, depending on whether there is or is not a pretreatment (hydrogen reduction) for 1 hour.

FIG. 2 schematically shows the conversion rates and selectivities of catalysts on which cobalt, zinc, platinum, and cobalt-zinc-platinum are respectively supported.

FIG. 3 schematically shows the conversion rates and selectivities of catalysts on which cobalt-zinc and cobalt-zinc-platinum are respectively supported.

FIG. 4 schematically shows the conversion rate, selectivity, and yield of the catalyst in which the amount of platinum supported on the Co—Zn catalyst is changed.

FIG. 5 schematically shows the conversion rates and selectivities of the catalysts prepared according to the two preparation methods of the present disclosure.

FIG. 6 schematically shows the conversion rates, selectivities, and yields with various reaction temperatures of the 4Co-8Zn-0.01Pt catalyst.

FIG. 7 schematically shows the conversion rates, selectivities and yields of the 4Co-8Zn-0.01Pt catalyst with various feed flow rates.

FIG. 8 schematically shows the conversion rates, selectivities and yields of the catalyst according to the number of recycles in the continuous reaction-regeneration.

### DETAILED DESCRIPTION

The catalyst for the production of olefins from alkane gases according to the present disclosure is one in which precursor solutions of cobalt, zinc, and platinum are co-impregnated and supported on alumina.

Hereinafter, preferred embodiments of the present disclosure will be described with reference to the accompanying drawings. However, the embodiments of the present disclosure may be modified in various other forms, and the scope of the present disclosure is not limited to the embodiments described below.

In describing the present embodiments, the same names and reference numerals are used for the same components, and thus overlapping additional descriptions are omitted below. In the drawings referenced below, no scale ratio applies.

The catalyst for the production of olefins from alkane gases according to the present disclosure is one in which precursor solutions of cobalt, zinc, and platinum are co-impregnated and supported on alumina.

The alumina support preferably has a y to 0 phase at a preparation temperature of 550 to 850° C., which is not less than the dehydrogenation reaction temperature, and has a surface area of 80 to 300 m²/g in this range.

When the support is prepared at a temperature lower than the dehydrogenation reaction temperature, thermal deformation of the catalyst may occur during the dehydrogenation reaction, and when it is prepared at a temperature exceeding 900° C., it has a low catalyst surface area due to crystallization of the carrier, and this inhibits mass transfer for catalytic activity upon contact with a reactant.

Traditionally, active metals for dehydrogenation catalysts vary, but cobalt is preferable to obtain high selectivity in the very early stage of the reaction within a few seconds, which is characteristic of the FPDH process. Further, it is preferable to add zinc and platinum to improve the conversion rate with maintaining the high selectivity properties of the cobalt-based catalyst.

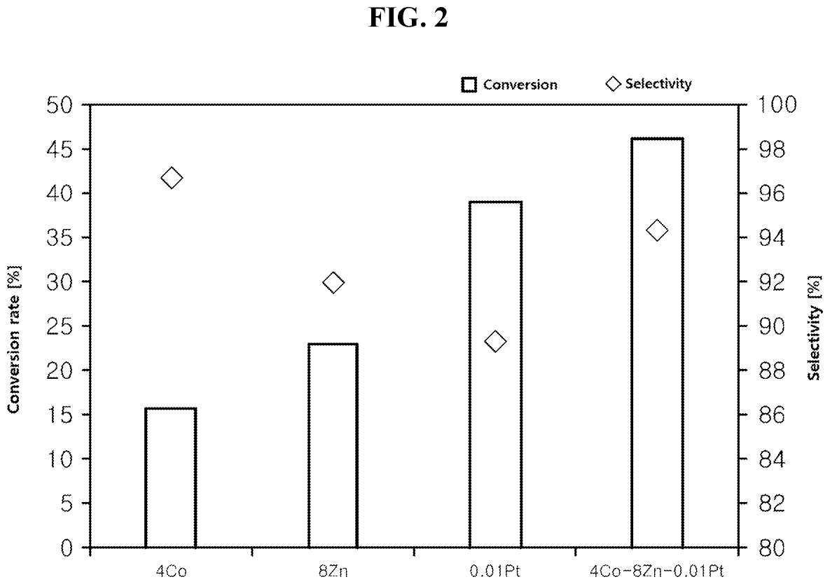

As shown in FIG. 2, the conversion rate within 1 to 3 seconds of TOS of the propane dehydrogenation reaction was shown to be the most contributed by platinum, and the highest selectivity was shown in the case of the cobalt catalyst. Therefore, in the 4Co-8Zn-0.01Pt catalyst system, it seems that propane conversion by platinum metal proceeds first, and it is estimated that the cobalt catalyst makes up for the low propylene selectivity due to the side reaction in the platinum catalyst. Furthermore, higher conversion rates and selectivity may be achieved by adding zinc.

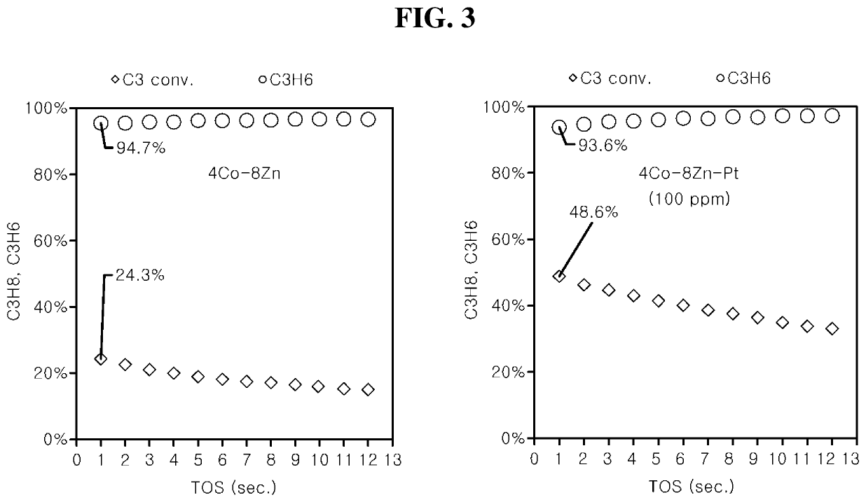

Further, as shown in FIG. 3, when the activity of the 4Co-8Zn catalyst was compared with that of the catalyst supported with 4Co-8Zn-0.01Pt to which 0.01% by weight of platinum was added, the conversion rate of the catalyst on which all three components were supported was increased by 24% or more, about 2 times, and the decrease in propylene selectivity was very insignificant, about 1%.

The catalyst is preferably calcinated at 700° C. to 900° C. The catalyst phase changes depending on the calcination temperature of the catalyst, and the catalyst is not preferable as a dehydrogenation catalyst since it forms a nano-sized crystalline phase outside the above temperature range so that it mainly causes a redox reaction.

It is preferable that cobalt is supported in an amount of 1 to 5% by weight based on the total catalyst weight. A catalyst amount outside the above range is outside the commercially applicable range for FPDH. Further, since a crystalline oxide is formed when the catalyst amount is large, the catalyst is negative as a dehydrogenation catalyst. Furthermore, when the catalyst amount is increased beyond the above range, the yield is significantly reduced.

It is preferable that zinc is supported in an amount of 2 to 10% by weight based on the total catalyst weight. As the amount of zinc increases, the conversion rate increases without changing the selectivity, but since the conversion rate decreases as the amount of zinc exceeds 10% by weight, the above range is preferable from a commercial point of view.

It is preferable that platinum is supported in an amount of 0.001 to 0.05% by weight based on the total catalyst weight.

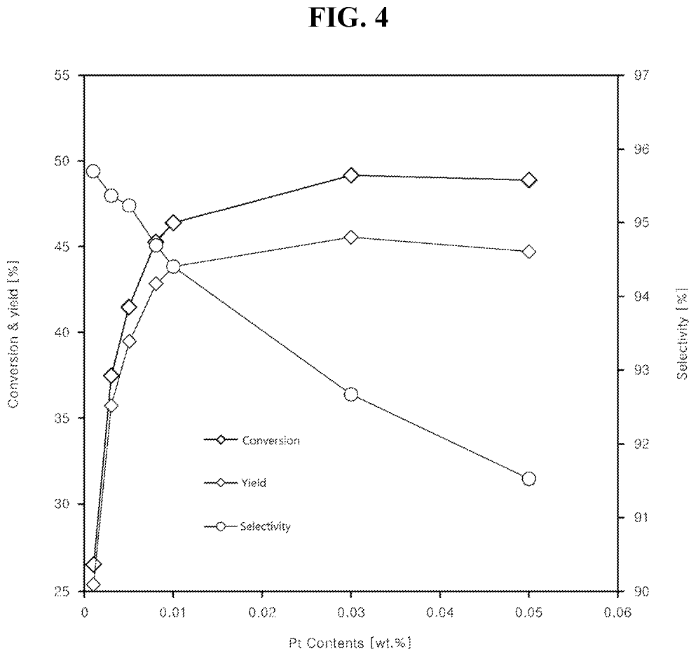

As shown in FIG. 4, when the amount of platinum supported on the Co—Zn catalyst was changed, the propane conversion rate rapidly increased when the amount of platinum was increased from 10 to 100 ppm, and increase in the conversion rate gradually increased after 100 ppm. The propylene selectivity decreased continuously as the amount of platinum increased.

Specifically, it can be seen that as the amount of platinum increases, the overall propylene yield also increases while the propane conversion rate increases. However, as the amount of platinum increases, side reactions also continuously increase, and the main by-products were methane and ethane. This indicates that the platinum catalyst has very high activity not only in the dehydrogenation reaction but also in the hydrogenolysis reaction in which produced hydrogen and propane meet to form methane and ethane.

Therefore, when considering the increase section in the conversion rate and the continuous decrease in the selectivity according to the amount of platinum introduced, it can be seen that one in which about 0.01% by weight (100 ppm) of platinum is combined with the 4Co-8Zn catalyst is the most suitable catalyst to be applied to the fast circulating fluidized bed process.

Meanwhile, it is preferable that the preparation method of a catalyst for the production of olefins from alkane gases, according to the present disclosure, comprises the steps of:

preparing a mixed solution by mixing cobalt, zinc and platinum precursors with water;

preparing a supported catalyst by impregnating the mixed solution on alumina;

drying the supported catalyst; and

calcinating the dried supported catalyst at 700° C. to 900° C.

It is preferable that another preparation method of a catalyst for the production of olefins from alkane gases, according to the present disclosure, comprises the steps of:

preparing a mixed solution by mixing cobalt and zinc precursors with water;

preparing a supported catalyst A by impregnating the mixed solution on alumina;

preparing a platinum precursor solution;

preparing a supported catalyst B by impregnating the platinum precursor solution on the supported catalyst A;

drying the supported catalyst B; and

calcinating the dried supported catalyst B at 700° C. to 900° C.

Conventionally, catalysts synthesized by the sol-gel method and the precipitation method, which are expected to have high crystallinity, are not preferable since CO2 production by oxidation reaction rather than dehydrogenation reaction is mainly performed. Meanwhile, in the case of a mesoporous catalyst with EISA method, which is a synthesis method with an increased alumina ratio, or a catalyst synthesized by a precipitation method on an alumina solid slurry, the acid site of the alumina support is appropriately controlled, and thus, the selectivity of the dehydrogenation reaction may be increased.

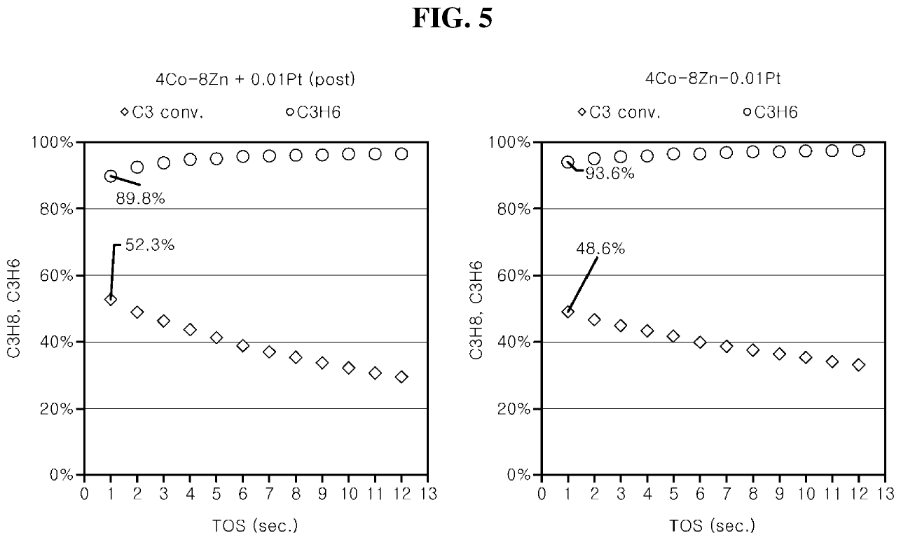

According to FIG. 5 showing the conversion rates and selectivity of the catalysts prepared according to the two preparation methods of the present disclosure, the 4Co-8Zn+0.01Pt(Post) catalyst is a catalyst in which platinum is additionally supported after a cobalt-zinc based catalyst is prepared, and the 4Co-8Zn-0.01Pt catalyst refers to a catalyst in which the aqueous precursor is supported on an alumina support after making cobalt-zinc-platinum into an aqueous precursor together. In the case of the catalyst in which platinum was added later, the most excellent conversion rate was shown due to the addition of the activity of the cobalt-zinc based catalyst and the high activity of platinum, whereas the initial selectivity was not significantly improved. As a result, it could be seen that the selectivity was greatly improved when the three metal precursors were supported together.

Another aspect of the present disclosure is to provide a method for the production of continuous reaction-regenerated olefins comprising a catalyst for the production of olefins from alkane gases produced according to the present disclosure. More preferably, it is to produce propylene from propane.

In the method for the production of continuous reaction-regenerated olefins, the reaction temperature is preferably 560 to 620° C.

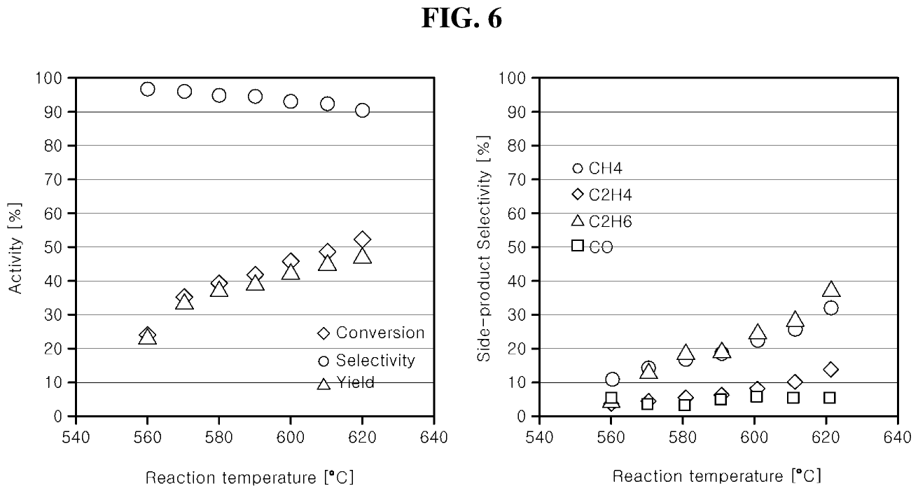

As shown in FIG. 6, as the reaction temperature increased, both the reaction activity and yield increased, but amount of methane and ethane production also increased, thereby showing a trend that the selectivity continued to decrease. Therefore, it is determined that the conversion rate is about 49% and the selectivity is 93% at 610° C., which is the most suitable state for the FPDH process.

In the method for the production of continuous reaction-regenerated olefins, it is preferable that the flow rate (WHSV) of alkanes as a feed is 4 to 16 h⁻¹.

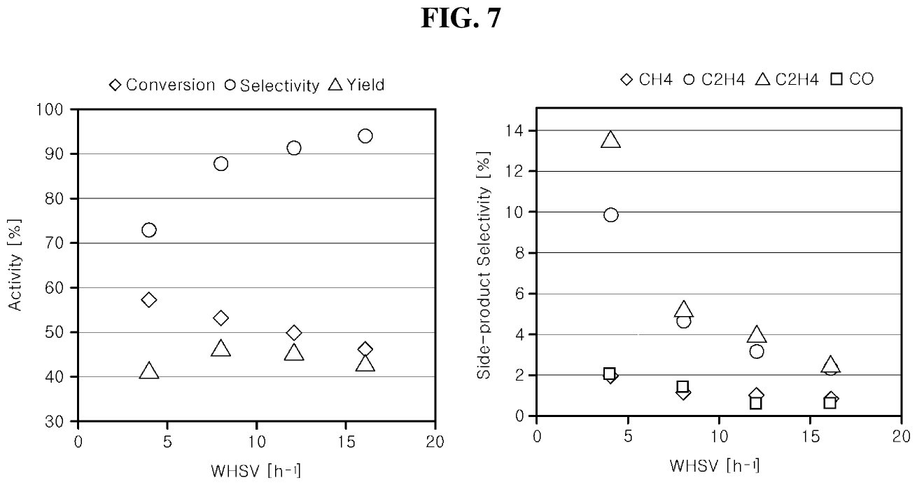

As shown in FIG. 7, as the flow rate (WHSV) decreased from 16 h⁻¹ to 4 h⁻¹, the time of contact with the catalyst increased so that the conversion rate increased linearly. Propylene selectivity decreased linearly up to WSHV 8 h⁻¹, and decreased sharply from 4 h⁻¹, and this is presumed to be due to the production of methane and ethane, which are platinum-based by-products.

Hereinafter, the present disclosure will be described in more detail through Preparation Example and Example.

### Preparation Example

1. Preparation of Platinum Alumina Catalyst (Pt/Alumina)

In order to prepare a metal oxide solution, water was prepared in a volume equal to the pore volume of alumina. A platinum oxide solution was prepared by dissolving H₂PtCl₆.xH2O (chloroplatinic acid) containing 10 ppm to 1,000 ppm (0.001 to 0.1% by weight) of platinum compared to alumina in prepared water. The prepared metal oxide solution was added to alumina, impregnated by incipient wetness impregnation, dried at 50 to 75° C. for 12 hours, and then calcinated in 700° C. to 900° C. with raising rate of 1° C. per minute for 6 hours to prepare a platinum alumina catalyst.

2. Preparation of Cobalt-Zinc-Platinum Alumina Catalysts (Co—Pt/Alumina, Zn—Pt, Co—Zn—Pt/Alumina) Through Co-Impregnation

In order to prepare a metal oxide solution, water was prepared in an amount equal to the alumina pore volume. Co(NO₃)₂.6H₂O (cobalt nitrate hexahydrate) containing 0 to 10% by weight of cobalt compared to alumina and Zn(NO₃)₂.6H₂O (zinc nitrate hexahydrate) containing 0 to 20% by weight of zinc metal), and finally H₂PtCl₆.xH₂O (chloroplatinic acid) containing 0 to 100 ppm (0 to 0.01% by weight) of platinum were subjected to co-impregnation to prepare cobalt-platinum, zinc-platinum, cobalt-zinc-platinum oxide solutions.

The metal oxide solutions prepared above were each added to alumina, impregnated by incipient wetness impregnation, dried at 50 to 75° C. for 12 hours, and then calcinated at a calcination temperature of 700° C. to 900° C. and a temperature raising rate of 1° C. per minute for 6 hours to prepare cobalt-zinc (0% by weight of platinum), cobalt-platinum (0% by weight of zinc), zinc-platinum (0% by weight of cobalt), and cobalt-zinc-platinum alumina catalysts respectively.

3. Preparation of Platinum-added Cobalt-Zinc Alumina Catalyst (Co—Zn/Alumina+Pt)

In order to investigate the catalytic activity according to the impregnation sequence of platinum, the cobalt-zinc alumina catalyst was separately impregnated with platinum, unlike the co-impregnation method in Preparation Example 2. First, in order to prepare a metal oxide solution, water was prepared in the same volume as the pore volume of alumina. A platinum oxide solution was prepared by dissolving H₂PtCl₆.xH2O (chloroplatinic acid) containing 10 to 100 ppm (0.001 to 0.01% by weight) of platinum compared to the cobalt-zinc alumina catalyst prepared through the co-impregnation method in Preparation Example 2 in water.

The prepared platinum oxide solution was added to the cobalt-zinc alumina catalyst prepared through the co-impregnation method in Preparation Example 2, impregnated by incipient wetness impregnation, dried at 50 to 75° C. for 12 hours, and then calcinated at a calcination temperature of 700° C. to 900° C. and a temperature raising rate of 1° C. per minute for 6 hours to prepare a cobalt-zinc-platinum alumina catalyst.

<Continuous Reaction Regeneration Experiment Method (Recycle Test) and Activity Evaluation>

After the prepared catalyst was injected into a fixed-bed type reactor using an automatic continuous reaction system equipped for continuous reaction-regeneration process, the temperature reached up to 600° C., which is a reaction and regeneration temperature, at a temperature raising rate of 10° C. per minute in an atmosphere of nitrogen gas that is an inert gas. After the reactor reached 600° C., a continuous reaction-regeneration experiment was performed. After nitrogen was flown into the reactor at a rate of 100 mL/min for 5 minutes, reduction was performed with 50 mL/min of a 50% propane/50% nitrogen mixed gas for 30 seconds. After nitrogen was flown into the reactor for 5 minutes again, the regeneration process was performed in an air atmosphere of 100 mL/min for 9 minutes and 30 seconds. This process was used as one reaction-regeneration experiment, and continuous regeneration was performed 1 to 1,000 times.

After recovering the catalyst from the continuous reaction regenerator and injecting 0.4 g of the prepared catalyst into a fixed-bed type reactor, the temperature reached up to 600° C., which is a reaction and regeneration temperature, at a temperature raising rate of 10° C. per minute in an atmosphere of helium gas that is an inert gas. Thereafter, reduction was performed with 105 mL/min of a 50% propane/50% nitrogen mixed gas for 16 seconds, and the regeneration process was performed in an air atmosphere of 30 mL/min. Next, after removing oxygen adsorbed to the reactor and the catalyst for 20 minutes using helium gas, a 50% propane/50% nitrogen mixed gas was injected at a flow rate of 105 mL/min to perform the reaction at a WHSV of 16h⁻¹. The reaction product was collected every second in the 16-port valve and analyzed through gas chromatography.

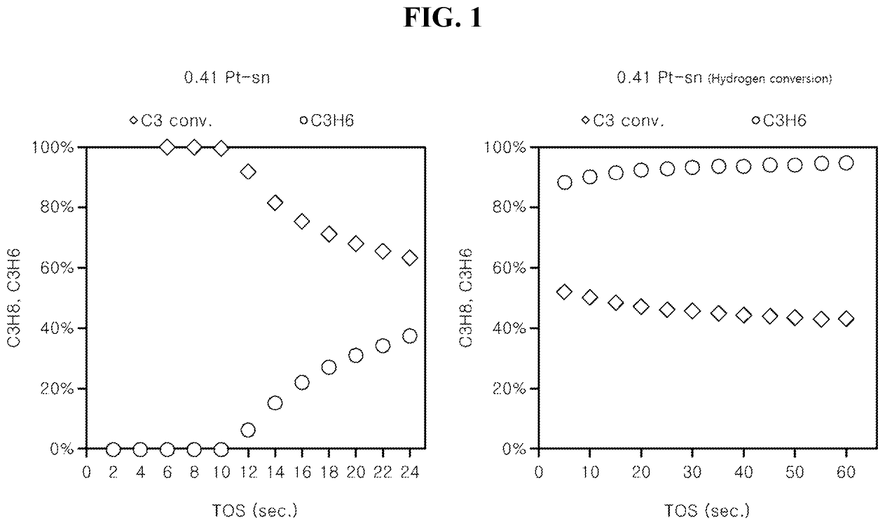

The results of experimenting the catalyst prepared above according to the continuous reaction-regeneration process are schematically shown in FIGS. 1 to 8.

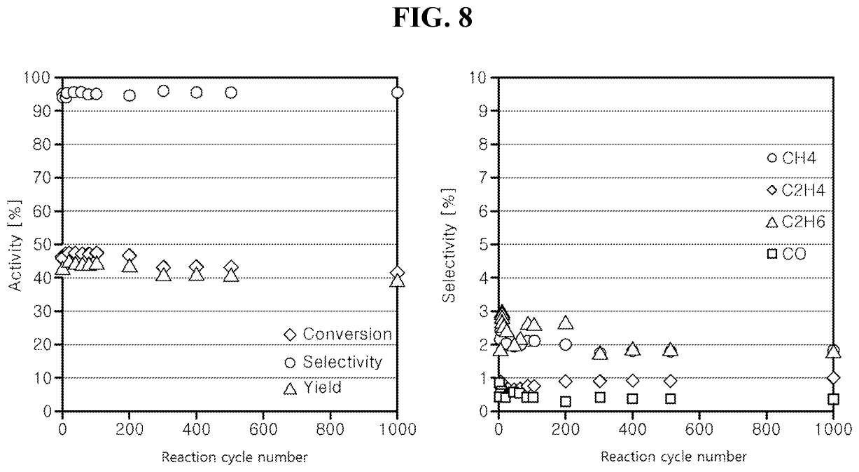

Particularly, as shown in FIG. 8, when looking at the catalyst activity according to the number of recycles in continuous reaction-regeneration process, a large change in conversion rates and selectivity could not be observed up to about 200 cycles of recycles (conversion rate range of 46 to 47% and selectivity range of 93 to 94%). However, the conversion rate decreased by about 3% and the selectivity increased to 95% from 300 cycles. Thereafter, the conversion rate and selectivity were maintained up to 500 cycles. Although deactivation of the catalyst was carried out from 300 cycles, it was confirmed that the conversion rate and selectivity were maintained in that state.

It could be confirmed that the catalyst according to the present disclosure exhibited a conversion rate of about 48% and a selectivity of 93% under conditions in which continuous reaction-regeneration process was possible without an additional hydrogen reduction process, despite the fact that platinum was added in an amount about 40 times less than the conventional catalyst.

This indicates that even with the same metal component of the dehydrogenation catalyst according to the reaction process, the effect varies depending on the optimal composition and loading amount of the combined catalyst. It could be found that the effect was excellent even with an extremely small amount of platinum required in the FPDH process compared to the amount required in the moving bed type process. Propylene selectivity was also greatly improved due to the introduction of a cobalt-zinc and the use of a trace amount of platinum.

Although the embodiments of the present disclosure have been described in detail above, these embodiments are exemplary, and the right scope of the present disclosure is not limited thereto. It will be apparent to those with ordinary skill in the art that various modifications and variations are possible without departing from the technical spirit of the present disclosure described in the claims.

1. A dehydrogenation catalyst for the production of olefins from alkane gases, in which cobalt, zinc and platinum precursor solutions are co-impregnated and supported on alumina.

2. The dehydrogenation catalyst of claim 1, wherein the catalyst is calcinated at 700° C. to 900° C.

3. The dehydrogenation catalyst of claim 1, wherein cobalt is supported in an amount of 1 to 5% by weight based on the total catalyst weight.

4. The dehydrogenation catalyst of claim 1, wherein zinc is supported in an amount of 2 to 10% by weight based on the total catalyst weight.

5. The dehydrogenation catalyst of claim 1, wherein platinum is supported in an amount of 0.001 to 0.05% by weight based on the total catalyst weight.

6. A preparation method of a dehydrogenation catalyst for the production of olefins from alkane gases, the preparation method comprising the steps of:
preparing a mixed solution by mixing cobalt, zinc and platinum precursors with water;
preparing a supported catalyst by impregnating the mixed solution on alumina;
drying the supported catalyst; and
calcinating the dried supported catalyst at 700° C. to 900° C.

7. A preparation method of a dehydrogenation catalyst for the production of olefins from alkane gases, the preparation method comprising the steps of:
preparing a mixed solution by mixing cobalt and zinc precursors with water;
preparing a supported catalyst A by impregnating the mixed solution on alumina;
preparing a platinum precursor solution;
preparing a supported catalyst B by impregnating the platinum precursor solution on the supported catalyst A;
drying the supported catalyst B; and
calcinating the dried supported catalyst B at 700° C. to 900° C.

8. The preparation method of claim 6, wherein cobalt is supported in an amount of 1 to 5% by weight based on the total catalyst weight.

9. The preparation method of claim 6, wherein zinc is supported in an amount of 2 to 10% by weight based on the total catalyst weight.

10. The preparation method of claim 6, wherein platinum is supported in an amount of 0.001 to 0.05% by weight based on the total catalyst weight.

11. A method for producing olefins in continuous reaction-regeneration process containing the catalyst of claim 1.

12. The method of claim 11, wherein the reaction temperature is 560 to 620° C.

13. The method of claim 11, wherein alkanes as a raw material in the olefin production method have a flow rate (WHSV) of 4 to 16 h⁻¹.

14. The preparation method of claim 7, wherein cobalt is supported in an amount of 1 to 5% by weight based on the total catalyst weight.

15. The preparation method of claim 7, wherein zinc is supported in an amount of 2 to 10% by weight based on the total catalyst weight.

16. The preparation method of claim 7, wherein platinum is supported in an amount of 0.001 to 0.05% by weight based on the total catalyst weight.
